(19) United States
(12) Patent Application Publication
(10) Pub. No.: US 2025/0099055 A1
(43) Pub. Date: Mar. 27, 2025

(54) INFORMATION PROCESSING APPARATUS, INFORMATION PROCESSING METHOD, AND PROGRAM

(71) Applicant: FUJIFILM CORPORATION, Tokyo (JP)

(72) Inventors: Hisatsugu HORIUCHI, Tokyo (JP); Takeru SUGIYAMA, Tokyo (JP); Ryoji SASADA, Tokyo (JP); Takeyasu KOBAYASHI, Tokyo (JP); Tomohiro NOMURA, Tokyo (JP)

(21) Appl. No.: 18/815,862

(22) Filed: Aug. 27, 2024

(30) Foreign Application Priority Data
Sep. 21, 2023 (JP) 2023-156461

(51) Int. Cl. A61B 6/46; A61B 6/00; A61B 6/50; G10L 15/22; G16H 30/40

(52) U.S. Cl. CPC A61B 6/468; A61B 6/463; A61B 6/487; A61B 6/50; G16H 30/40; G10L 15/22

(57) ABSTRACT

The information processing apparatus includes at least one processor. The processor acquires a series of radiation images captured by performing continuous irradiation with radiation, acquires relevant information that is generated by recognizing a voice and is related to imaging content of the series of radiation images, and associates the series of radiation images with the relevant information.

### CROSS-REFERENCE TO RELATED APPLICATIONS

The present application claims priority under 35 U.S.C. § 119 to Japanese Patent Application No. 2023-156461, filed on Sep. 21, 2023. The above application is hereby expressly incorporated by reference, in its entirety, into the present application.

### BACKGROUND

### 1. Technical Field

The disclosed technology relates to an information processing apparatus, an information processing method, and a program.

### 2. Description of the Related Art

The following technologies are known as technologies relating to image processing using a voice. For example, JP2014-036682A discloses an endoscope apparatus comprising an imaging unit that images a subject and outputs an image signal, a voice acquisition unit that records a voice and outputs a voice signal, a character generation unit that receives the voice signal and converts the voice signal into a character string to generate a character signal, and a video creation unit that receives the image signal, the voice signal, and the character signal and creates a video file. The video creation unit records the character signal as a character code in the video file separately from the image signal.

JP2007-082088A discloses an apparatus for converting content including any of a video, a voice, or data into a stream, and recording and playing back the stream in an information recording medium together with metadata related to the video, the voice, or the data. The apparatus comprises a voice recognition unit that selects an externally input voice and converts the externally input voice into character data using voice recognition, and by setting a recording control unit, the character data or information obtained by adding format information to the character data is inserted into a data unit constituting a video frame, thereby recording metadata or character data marking a scene in the information recording medium as metadata related to the content.

### SUMMARY

A videofluoroscopic examination of swallowing is known as one type of examination using radiation images. A videofluoroscopic examination of swallowing is an examination to evaluate a process and a state when an examinee swallows a sample. In a videofluoroscopic examination of swallowing, a video composed of a series of radiation images captured by continuously irradiating an examinee with radiation is used. In a videofluoroscopic examination of swallowing, images of an examinee swallowing a sample are captured while appropriately changing the combination of the examinee's posture and the type of sample (that is, while changing the examination level). In such an examination using radiation images, in a case in which a plurality of examination levels exist, it is necessary to accurately associate the contents of the examination level with the captured radiation image so that it is possible to ascertain which examination level the captured radiation image corresponds to.

In videofluoroscopic examinations of swallowing in the related art, a lead marker engraved with the type of sample, such as, for example, “thick liquid”, “chopped food”, and “porridge” was imaged together with an examinee. However, the task of disposing the marker at a position that does not overlap the examinee's region of interest is time-consuming, which leads to a longer examination time. In addition, it was necessary for an imaging person, such as a doctor or a radiologist, to position the marker, which results in exposure to radiation for the imaging person.

The disclosed technology has been made in consideration of the above-mentioned points, and an object thereof is to easily and accurately associate relevant information related to imaging content of a radiation image with the radiation image.

According to an aspect of the disclosed technology, there is provided an information processing apparatus comprising at least one processor. The processor is configured to: acquire a series of radiation images captured by performing continuous irradiation with radiation; acquire relevant information that is generated by recognizing a voice and is related to imaging content of the series of radiation images; and associate the series of radiation images with the relevant information.

The processor may be configured to associate the latest relevant information acquired before a predetermined timing synchronized with the capturing of the series of radiation images with the series of radiation images. The predetermined timing may be a timing of start of irradiation with the radiation that is continuously performed in the capturing of the series of radiation images. The predetermined timing may be confirmed by receiving a radiation switch signal, which is transmitted by a user input unit and indicates the start of irradiation that is continuously performed in the capturing of the series of radiation images.

The processor may be configured to generate a data file including the relevant information and image group data including the series of radiation images. The processor may include a notation corresponding to the relevant information in a file name of a data file including the series of radiation images. The processor may be configured to process image group data including the series of radiation images so that a notation corresponding to the relevant information is displayed together with the series of radiation images.

The processor may be configured to associate, in a case in which new relevant information generated by recognizing a voice uttered during an imaging period of the series of radiation images is acquired, the new relevant information with a radiation image acquired after the acquisition of the new relevant information from among the series of radiation images.

The processor may be configured to display a radiation image associated with designated relevant information from among the series of radiation images. The processor may be configured to: acquire relevant information generated by a method other than voice recognition; and replace the relevant information generated by the voice recognition with the relevant information generated by the method other than the voice recognition. The processor may be configured to issue an alert in a case in which the relevant information associated with the series of radiation images does not include specific information.

The relevant information may be information indicating a type of sample used in an examination using the series of radiation images. The relevant information may be information indicating a type of posture of an examinee in an examination using the series of radiation images. The relevant information may be information indicating a type of imaging direction of the series of radiation images. The examination may be a videofluoroscopic examination of swallowing. In videofluoroscopic examinations of swallowing, the burden of supporting the examinee is large. Therefore, by applying the disclosed technology to videofluoroscopic examinations of swallowing, workability can be improved. The relevant information may be generated by recognizing a voice by which a predetermined term is pronounced.

According to another aspect of the disclosed technology, there is provided an information processing method executed by at least one processor included in an information processing apparatus, the information processing method comprising: acquiring a series of radiation images captured by performing continuous irradiation with radiation; acquiring relevant information that is generated by recognizing a voice and is related to imaging content of the series of radiation images; and associating the series of radiation images with the relevant information.

According to still another aspect of the disclosed technology, there is provided a program for causing at least one processor included in an information processing apparatus to execute:

acquiring a series of radiation images captured by performing continuous irradiation with radiation; acquiring relevant information that is generated by recognizing voice and is related to imaging content of the series of radiation images; and associating the series of radiation images with the relevant information.

According to the aspects of the disclosed technology, it is possible to easily and accurately associate relevant information related to imaging content of a radiation image with the radiation image.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a radiography system according to an embodiment of the disclosed technology;

FIG. 2 is a diagram showing an example of a configuration of a radiography apparatus according to the embodiment of the disclosed technology;

FIG. 3 is a diagram showing an example of a hardware configuration of an information processing apparatus according to the embodiment of the disclosed technology;

FIG. 4 is a functional block diagram showing an example of a functional configuration of the information processing apparatus according to the embodiment of the disclosed technology;

FIG. 5 is a diagram showing an example of a structure of a data file generated by an association processing unit according to the embodiment of the disclosed technology;

FIG. 6 is a flowchart showing an example of a flow of processing performed by a CPU according to the embodiment of the disclosed technology executing an association program;

FIG. 7 is a diagram showing an example of association processing performed by the information processing apparatus according to the embodiment of the disclosed technology;

FIG. 8 is a functional block diagram showing an example of a functional configuration of an information processing apparatus according to another embodiment of the disclosed technology;

FIG. 9 is a flowchart showing an example of a flow of processing performed by a CPU according to the other embodiment of the disclosed technology executing an association program;

FIG. 10 is a diagram showing an example of association processing performed by the information processing apparatus according to the other embodiment of the disclosed technology;

FIG. 11 is a functional block diagram showing an example of a functional configuration of an information processing apparatus according to still another embodiment of the disclosed technology; and

FIG. 12 is a diagram showing a posture of an examinee in a videofluoroscopic examination of swallowing.

### DETAILED DESCRIPTION

An example of embodiments of the disclosed technology will be described below with reference to the drawings. In addition, the same or equivalent components and parts in each drawing are given the same reference numerals, and duplicated descriptions will be omitted.

### First Embodiment

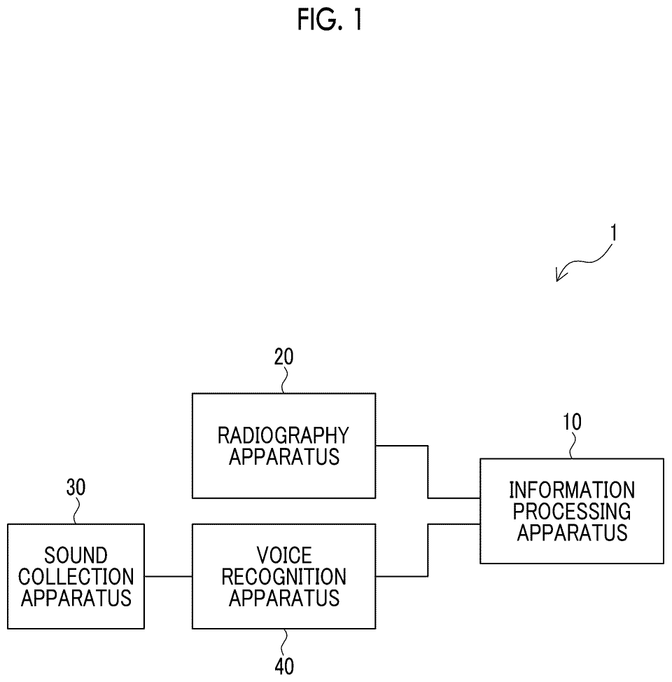

FIG. 1 is a diagram showing an example of a configuration of a radiography system 1 according to an embodiment of the disclosed technology. In the following description, an example in which the radiography system 1 is used for a videofluoroscopic examination of swallowing will be described. The radiography system 1 includes an information processing apparatus 10, a radiography apparatus 20, a sound collection apparatus 30, and a voice recognition apparatus 40.

The radiography apparatus 20 is capable of imaging a radiation image using radiation such as X-rays. It is also possible to sequentially display a series of radiation images captured by performing continuous irradiation with radiation in a time series manner, thereby producing a video display. The imaging of a series of radiation images for producing a video display is called fluoroscopy. In a videofluoroscopic examination of swallowing, fluoroscopy is performed to observe the process in which an examinee swallows a sample. Note that “continuous irradiation with radiation” includes both a case in which irradiation with radiation is continuously performed over a certain period of time and a case in which irradiation with pulsed radiation is performed a plurality of times within a certain period of time. The radiation image captured by the radiography apparatus 20 is immediately transmitted to the information processing apparatus 10.

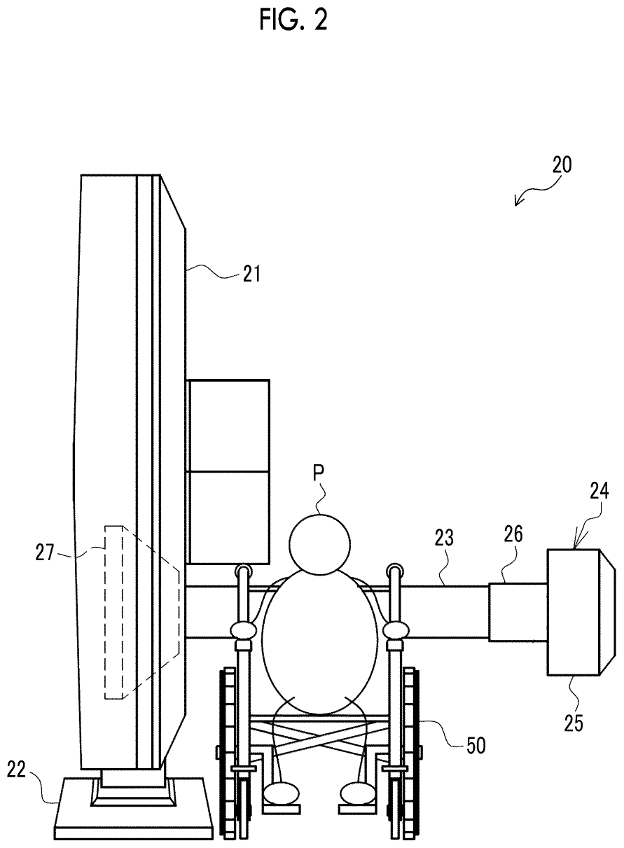

FIG. 2 is a diagram showing an example of the configuration of the radiography apparatus 20. The radiography apparatus 20 has an imaging table 21. The imaging table 21 is supported on the floor surface by a stand 22. A radiation generation unit 24 is attached to the imaging table 21 via a support column 23. The radiation generation unit 24 includes a radiation source 25 and a collimator 26. The imaging table 21 has a built-in radiation detector 27.

The radiation source 25 has a radiation tube (not shown) and irradiates an examinee P with radiation. The collimator 26 limits the irradiation field of radiation emitted from the radiation tube. The radiation detector 27 has a plurality of pixels that generate signal charges according to the radiation that has transmitted through the examinee P. The radiation detector 27 is called a flat panel detector (FPD).

The radiation generation unit 24 is capable of reciprocating together with the support column 23 along a long side direction of the imaging table 21 by a moving mechanism (not shown) such as a motor. The radiation detector 27 is capable of reciprocating along the long side direction of the imaging table 21 in conjunction with the movement of the radiation generation unit 24. The imaging table 21 and the support column 23 can be rotated between a standing state shown in FIG. 2 and a supine state (not shown) by a rotation mechanism (not shown) such as a motor. In the supine state, the surface of the imaging table 21 is parallel to the floor surface, and the direction in which the support column 23 extends is perpendicular to the floor surface. On the other hand, in the standing state, the surface of the imaging table 21 is perpendicular to the floor surface, and the direction in which the support column 23 extends is parallel to the floor surface. In the standing state, it is possible to perform fluoroscopy on the examinee P in a wheelchair 50 as shown in FIG. 2.

The sound collection apparatus 30 is a microphone that collects surrounding sounds. The sound collection apparatus 30 may be provided in the voice recognition apparatus 40, or may be a neck microphone or the like worn by an imaging person who captures a radiation image, such as a doctor or a radiologist. The uttered voice of an imaging person who captures a radiation image, such as a doctor or a radiologist, is collected by the sound collection apparatus 30. The voice recognition apparatus 40 recognizes the voice collected by the sound collection apparatus 30 using a known voice recognition technology. In a case in which the collected voice includes a predetermined specific term indicating the content of the examination level in the videofluoroscopic examination of swallowing, the voice recognition apparatus 40 generates identification information of the examination level indicated by the term as relevant information related to the imaging of the radiation image.

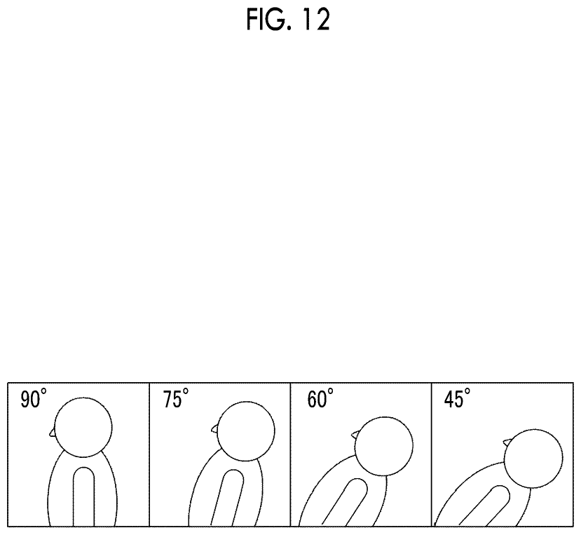

Specific terms indicating the content of the examination level may include terms indicating the type of sample that the examinee swallows in the videofluoroscopic examination of swallowing, such as “water”, “thick liquid”, “chopped food”, and “porridge”. In this case, the relevant information includes identification information of the type of sample. In addition, specific terms indicating the content of the examination level may include terms indicating the type of posture taken by the examinee during the videofluoroscopic examination of swallowing, such as “90°”, “75°”, “60°”, “45°”, “30°”, “0°”, “supine”, and “prone”. In this case, the relevant information includes identification information of the type of posture of the examinee. The above angle is the angle of the spine with the horizontal direction as the reference (0°) in a case in which the examinee swallows in a videofluoroscopic examination of swallowing, as shown in FIG. 12. In addition, the specific terms indicating the content of the examination level may include terms indicating the type of imaging direction of the radiation images captured in the videofluoroscopic examination of swallowing, such as “frontal imaging” and “lateral imaging”. In this case, the relevant information includes identification information for the type of imaging direction.

For example, in a case in which the voice collected by the sound collection apparatus 30 includes the term “thick liquid”, which indicates the type of sample that is the content of the examination level, the voice recognition apparatus 40 generates identification information corresponding to “thick liquid” as relevant information. In this case, the voice recognition apparatus 40 may generate, as relevant information, for example, text describing “thick liquid” or a level code consisting of characters, numerical values, symbols, or a combination of these corresponding to “thick liquid”. In addition, in a case in which the voice collected by the sound collection apparatus 30 includes the term “forty five degrees”, which indicates the posture of the examinee that is the content of the examination level, the voice recognition apparatus 40 generates identification information corresponding to “45°” as relevant information. In this case, the voice recognition apparatus 40 may generate, as relevant information, for example, text describing “45°” or a level code consisting of characters, numerical values, symbols, or a combination of these corresponding to “45°”. The terms indicating the examination levels, such as “thick liquid”, “chopped food”, “porridge”, “forty five degrees”, and “ninety degrees” given as examples above, are registered in advance in the voice recognition apparatus 40. By using pre-registered terms as the target of voice recognition, it is possible to improve the accuracy of voice recognition. In a case in which the recognized voice includes a specific term, the voice recognition apparatus 40 instantly generates relevant information and immediately transmits the relevant information to the information processing apparatus 10. That is, the relevant information is transmitted to the information processing apparatus 10 immediately after the occurrence of a voice including a specific term. In a case in which an utterance including a specific term is made intermittently over a plurality of times, relevant information corresponding to each utterance is generated in sequence and transmitted to the information processing apparatus 10 in sequence.

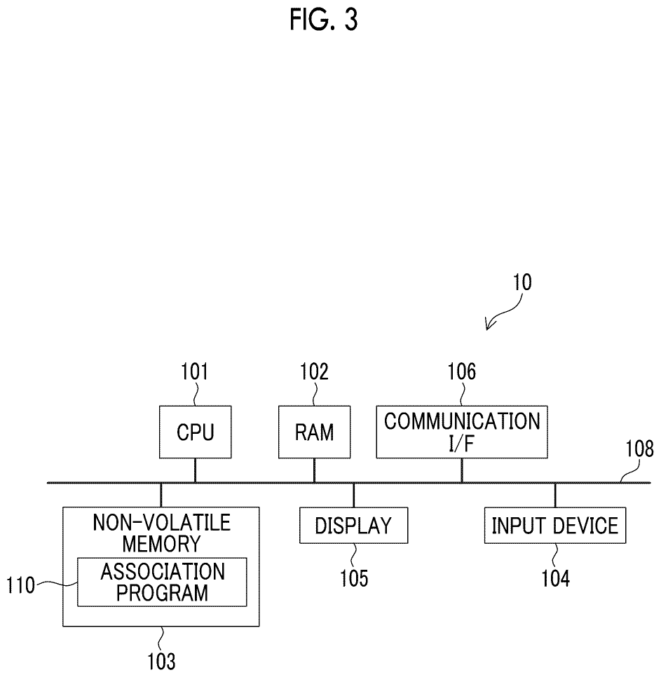

FIG. 3 is a diagram showing an example of a hardware configuration of the information processing apparatus 10. The information processing apparatus 10 includes a central processing unit (CPU) 101, a random-access memory (RAM) 102, a non-volatile memory 103, an input device 104 including a keyboard and a mouse, a display 105, and a communication interface 106. These pieces of hardware are connected to a bus 108.

The display 105 may be a touch panel display. The communication interface 106 is an interface for the information processing apparatus 10 to communicate with the radiography apparatus 20 and the voice recognition apparatus 40. The communication method may be either wired or wireless. For the wireless communication, it is possible to apply a method conforming to existing wireless communication standards such as, for example, Wi-Fi (registered trademark) and Bluetooth (registered trademark).

The non-volatile memory 103 is a non-volatile storage medium such as a hard disk or a flash memory. The non-volatile memory 103 stores an association program 110. The RAM 102 is a work memory for the CPU 101 to execute processing. The CPU 101 loads the association program 110 stored in the non-volatile memory 103 into the RAM 102, and executes processing in accordance with the association program 110. The CPU 101 is an example of a “processor” in the disclosed technology.

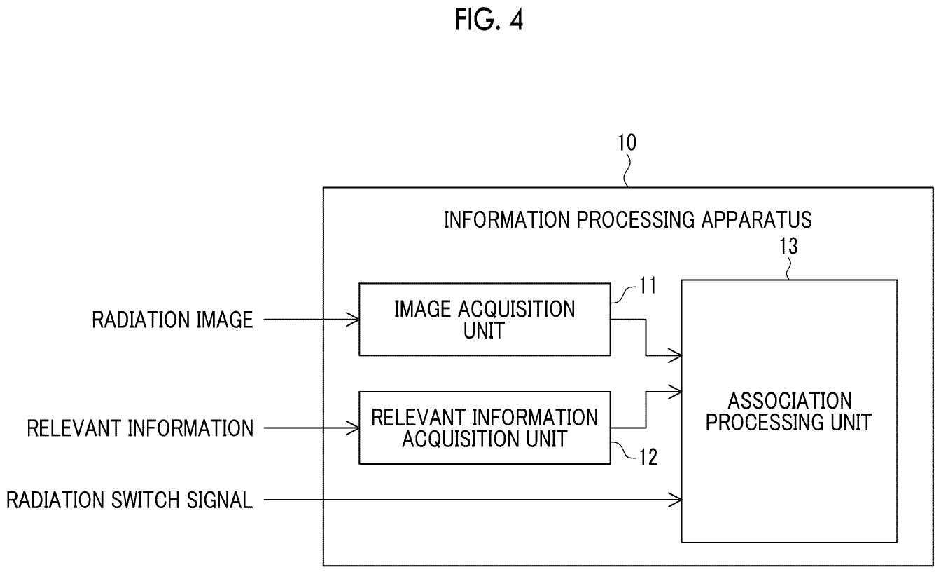

FIG. 4 is a functional block diagram showing an example of a functional configuration of the information processing apparatus 10. The information processing apparatus 10 includes an image acquisition unit 11, a relevant information acquisition unit 12, and an association processing unit 13. In a case in which the CPU 101 executes the association program 110, the information processing apparatus 10 functions as the image acquisition unit 11, the relevant information acquisition unit 12, and the association processing unit 13.

The image acquisition unit 11 acquires a series of radiation images captured by performing continuous irradiation with radiation in the radiography apparatus 20. A series of radiation images are obtained by fluoroscopically imaging the process in which an examinee chews and swallows a sample in a videofluoroscopic examination of swallowing.

The relevant information acquisition unit 12 acquires relevant information generated by the voice recognition apparatus 40. The relevant information is information related to the imaging content of the series of radiation images acquired by the image acquisition unit 11. Specifically, identification information of the examination level generated based on the utterance of a doctor or radiologist at the time of capturing a series of radiation images is acquired as relevant information. The relevant information is acquired immediately after the occurrence of a voice including a specific term. In a case in which a plurality of pieces of relevant information are generated intermittently, the relevant information is acquired by the relevant information acquisition unit 12 in sequence.

The association processing unit 13 associates the series of radiation images acquired by the image acquisition unit 11 with the relevant information acquired by the relevant information acquisition unit 12. Specifically, the association processing unit 13 associates the latest relevant information acquired before a predetermined timing synchronized with the capturing of the series of radiation images with the series of radiation images. The association processing unit 13 uses a radiation switch signal input to the information processing apparatus 10 to determine relevant information to be associated with each other from among relevant information that can be sequentially acquired.

The radiation switch signal is a signal that indicates the start and end of irradiation with radiation, and is, for example, a signal that is output in conjunction with the on/off of a foot switch operated by a doctor or radiologist at the time of the irradiation with radiation in the radiography apparatus 20. In the radiography system 1 according to the present embodiment, a common radiation switch signal is provided to the radiography apparatus 20 and the information processing apparatus 10. In the following description, it is assumed that the radiation switch signal exhibits a high level during irradiation periods with radiation (including non-irradiation periods in a case in which irradiation with pulsed radiation is performed a plurality of times) and exhibits a low level otherwise. The association processing unit 13 associates the latest relevant information acquired before the timing at which the radiation switch signal transitions to a high level with the series of radiation images. The timing at which the radiation switch signal transitions to a high level corresponds to the timing of start of irradiation with the radiation that is continuously performed in capturing a series of radiation images. In a case in which a plurality of pieces of relevant information have been acquired before a timing of the start of irradiation with radiation, the association processing unit 13 sets the latest relevant information among the plurality of pieces of relevant information acquired up to that timing as the target of association.

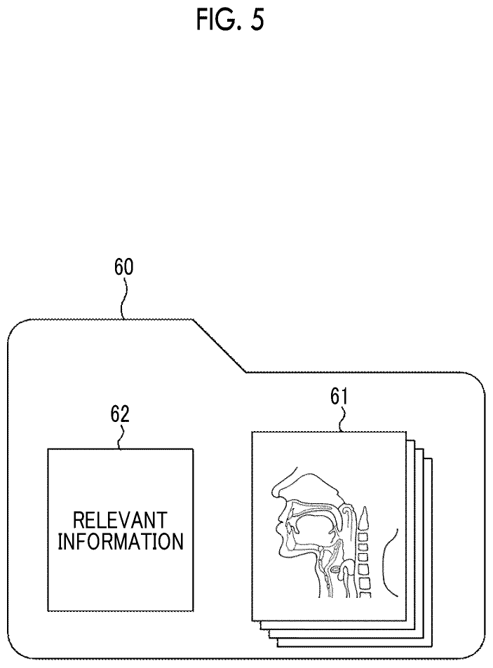

Various aspects can be exemplified as an aspect of the association between a series of radiation images and relevant information. For example, the association processing unit 13 may associate a series of radiation images with the relevant information by generating a data file 60 including relevant information 62 and image group data 61 including a series of radiation images, as shown in FIG. 5.

The association processing unit 13 may also associate a series of radiation images with the relevant information by including a notation corresponding to the relevant information in the file name of a data file including the series of radiation images. “Notation corresponding to relevant information” refers to characters, numbers, symbols, or a combination thereof that can identify the content of the examination level indicated by the relevant information. For example, characters such as “thick liquid” indicating the content of the examination level are applied as a portion of the file name of the data file.

Furthermore, the association processing unit 13 may process image group data including a series of radiation images so that notations corresponding to relevant information are displayed together with the series of radiation images. In this case, in a case in which a series of radiation images are displayed as a video, characters such as “thick liquid” indicating the content of the examination level indicated by the relevant information are displayed together with the video.

The association processing unit 13 stores a data file in which a series of radiation images and relevant information are associated with each other in a recording medium. The recording medium in which the data file is stored may be the non-volatile memory 103 included in the information processing apparatus 10, or may be a recording medium included in an external image server (not shown).

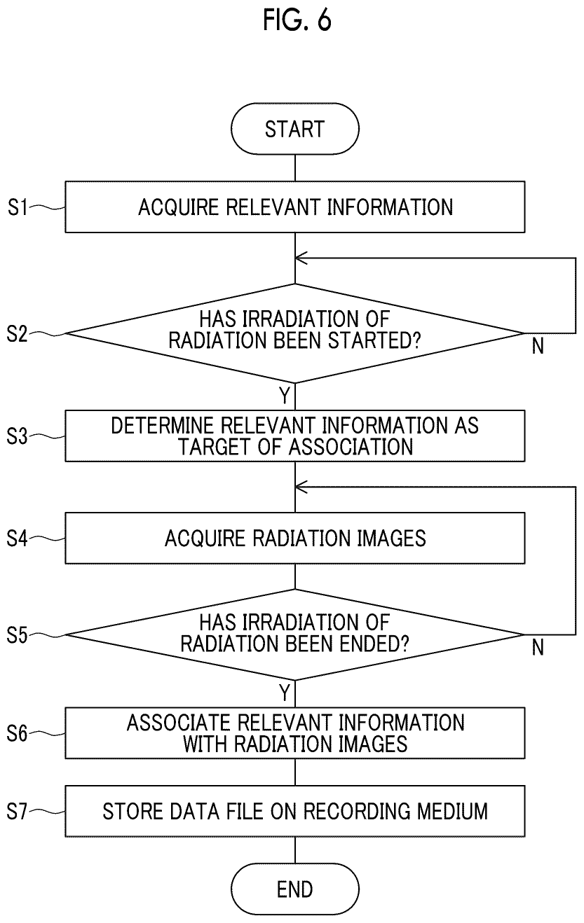

FIG. 6 is a flowchart showing an example of a flow of processing performed by the CPU 101 executing the association program 110.

In Step S1, the relevant information acquisition unit 12 acquires relevant information generated by the voice recognition apparatus 40. In a case in which a plurality of pieces of relevant information are generated intermittently by the voice recognition apparatus 40, the relevant information acquisition unit 12 acquires these pieces of relevant information in sequence.

In Step S2, the association processing unit 13 determines whether or not irradiation with radiation has been started in the radiography apparatus 20. In a case in which the radiation switch signal transitions from a low level to a high level, the association processing unit 13 determines that irradiation with radiation has started.

In Step S3, the association processing unit 13 determines the latest relevant information acquired before the start of irradiation with radiation as relevant information to be associated with a series of radiation images.

In Step S4, the image acquisition unit 11 acquires a series of radiation images captured by performing continuous irradiation with radiation in the radiography apparatus 20. A series of radiation images are obtained by fluoroscopically imaging the process in which an examinee swallows a sample in a videofluoroscopic examination of swallowing. The acquisition of a series of radiation images by the image acquisition unit 11 continues until the irradiation with radiation (fluoroscopy) ends in the radiography apparatus 20.

In Step S5, the association processing unit 13 determines whether or not the irradiation with radiation has been ended in the radiography apparatus 20. In a case in which the radiation switch signal transitions from a high level to a low level, the association processing unit 13 determines that irradiation with radiation has been ended.

In Step S6, the association processing unit 13 associates the relevant information determined as the target of association in Step S3 with the series of radiation images acquired in Step S4. The association processing unit 13 associates a series of radiation images with the relevant information by generating a data file 60 including relevant information 62 and image group data 61 including a series of radiation images, as shown in FIG. 5, for example.

In Step S7, the association processing unit 13 stores the data file generated in Step S6 in the recording medium.

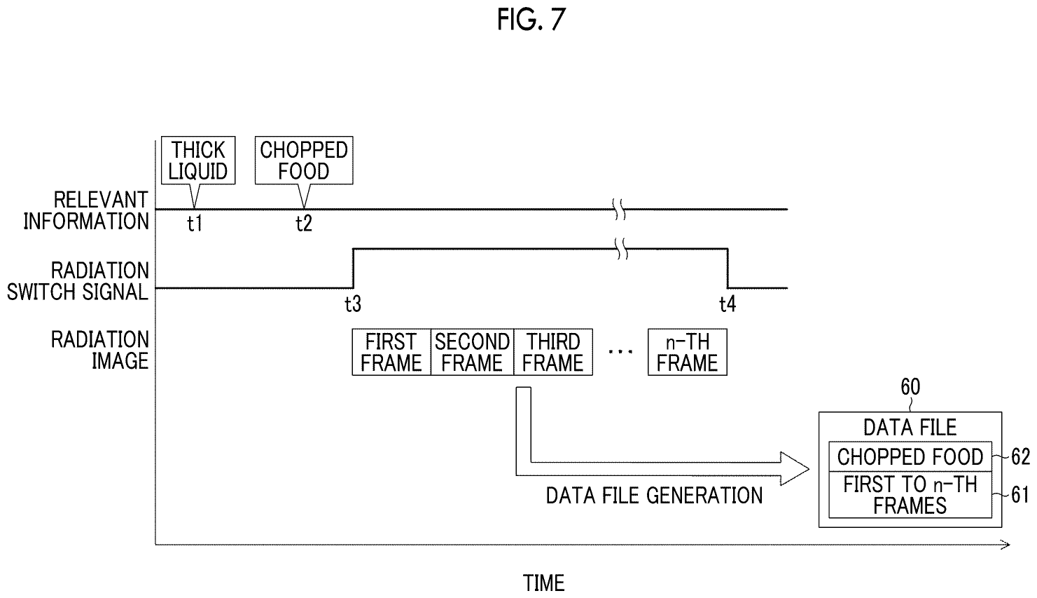

FIG. 7 is a diagram showing an example of association processing performed by the information processing apparatus 10. In a case in which an imaging person who captures a radiation image, such as a doctor or a radiologist, makes an utterance including the term “thick liquid” indicating the content of the examination level at time point t1, the voice recognition apparatus 40 generates identification information of the examination level corresponding to “thick liquid” (for example, text describing “thick liquid”) as relevant information. The relevant information is acquired immediately by the information processing apparatus 10.

In a case in which an imaging person who captures a radiation image, such as a doctor or a radiologist, makes an utterance including the term “chopped food” indicating the content of the examination level at time point t2, the voice recognition apparatus 40 generates identification information of the examination level corresponding to “chopped food” (for example, text describing “chopped food”) as relevant information. The relevant information is acquired immediately by the information processing apparatus 10.

At time point t3 at which the radiation switch signal transitions from a low level to a high level, the radiography apparatus 20 starts irradiating with radiation. In a videofluoroscopic examination of swallowing, a series of radiation images is captured by performing continuous irradiation with radiation. A series of radiation images constitute each frame in a case in which the series of radiation images are displayed as a video. A plurality of frames (radiation images) from the first frame to the n-th frame are generated in the radiography apparatus 20 until time point t4 at which the radiation switch signal transitions to a low level. The radiation image generated by the radiography apparatus 20 is acquired immediately by the information processing apparatus 10.

The information processing apparatus 10 determines, as the target of association, the relevant information corresponding to the latest “chopped food” acquired by time point t3 at which the radiation switch signal transitions to a high level. The information processing apparatus 10 generates a data file 60 including image group data 61 including a series of radiation images (first to n-th frames) and relevant information 62 corresponding to the “chopped food” and stores the data file 60 in a recording medium.

As described above, the information processing apparatus 10 according to the embodiment of the disclosed technology acquires a series of radiation images captured by performing continuous irradiation with radiation, acquires relevant information that is generated by recognizing a voice and is related to imaging content of the series of radiation images, and associates the series of radiation images with the relevant information. With the information processing apparatus 10 according to the present embodiment, the relevant information generated by recognizing the voice is associated with a series of radiation images, so that the user does not need to perform a special operation for the association. That is, the information processing apparatus 10 according to the present embodiment makes it possible to easily and accurately associate relevant information with a radiation image.

Furthermore, with the information processing apparatus 10 according to the present embodiment, the latest relevant information acquired before a predetermined timing synchronized with the capturing of the series of radiation images is associated with the series of radiation images. The predetermined timing is a timing of start of irradiation with the radiation that is continuously performed in capturing a series of radiation images.

In a case of associating relevant information generated in response to utterances by an imaging person who captures a radiation image, such as a doctor or a radiologist, with a series of radiation images, it is considered that utterances made immediately before the start of imaging include appropriate relevant information. By setting the latest relevant information acquired before the timing of starting irradiation with radiation as the target of association, it becomes possible to set appropriate relevant information as the target of association.

The information processing apparatus 10 may have the following additional functions. The information processing apparatus 10 may acquire relevant information generated by a method other than voice recognition, and replace the relevant information generated by the voice recognition with the relevant information generated by the method other than the voice recognition. According to this aspect, for example, in a case in which there is an error in relevant information generated by voice recognition, it is possible to update the erroneous relevant information with the relevant information generated by the method other than the voice recognition. The “relevant information generated by the method other than the voice recognition” may be, for example, relevant information generated by operating an input device such as a keyboard or a function button.

Furthermore, the information processing apparatus 10 may issue an alert in a case in which the relevant information associated with the series of radiation images does not include specific information. The “specific information” may be identification information of the examination level to be included in the videofluoroscopic examination of swallowing. According to this aspect, it is possible to notify that radiation images relating to the examination level to be included in the videofluoroscopic examination of swallowing have not yet been captured.

### Second Embodiment

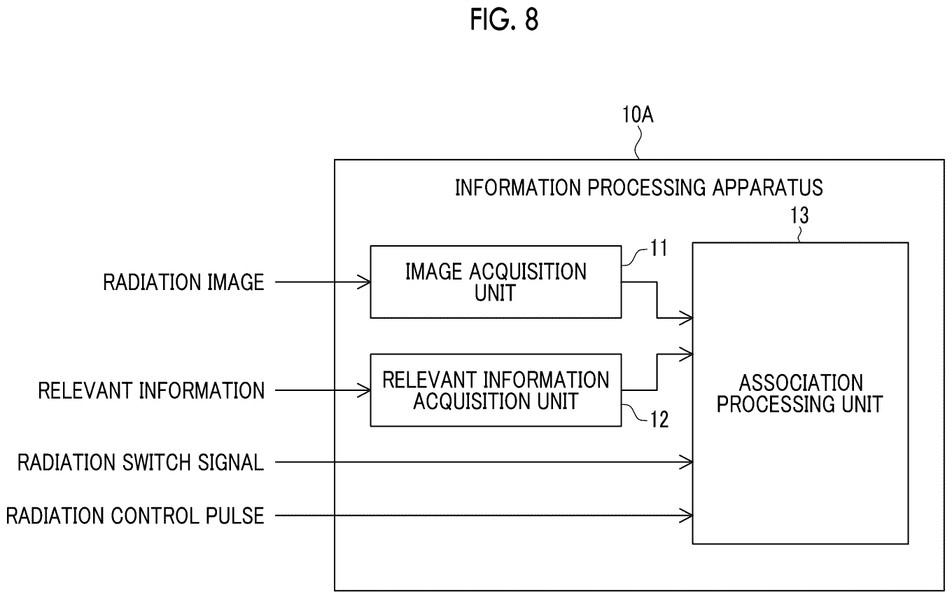

FIG. 8 is a functional block diagram showing an example of a functional configuration of an information processing apparatus 10A according to a second embodiment of the disclosed technology. In the information processing apparatus 10 according to the first embodiment, one piece of relevant information is associated with a series of radiation images. In contrast, the information processing apparatus 10A according to the second embodiment associates a plurality of pieces of relevant information with a series of radiation images. More specifically, the association processing unit 13 according to the second embodiment associates, in a case in which new relevant information generated by recognizing a voice uttered during an imaging period of the series of radiation images is acquired, the new relevant information with a radiation image acquired after the acquisition of the new relevant information from among the series of radiation images. With the information processing apparatus 10A according to the present embodiment, certain relevant information is associated with some of frames of a video composed of a series of radiation images, and different relevant information is associated with some of the other frames.

In the information processing apparatus 10A according to the second embodiment, the association processing unit 13 uses a radiation control pulse input to the information processing apparatus 10 to associate relevant information with each radiation image (frame). In the present embodiment, the radiography apparatus 20 captures a series of radiation images by performing irradiation with pulsed radiation a plurality of times within a certain period of time. The radiation control pulse is a pulse signal corresponding to each pulse of the pulsed radiation. That is, one pulse of the radiation control pulse corresponds to one pulse of the pulsed radiation. One radiation image (one frame) is generated by performing irradiation with one pulse of radiation. Therefore, one pulse of the radiation control pulse corresponds to one radiation image (one frame) of the series of radiation images.

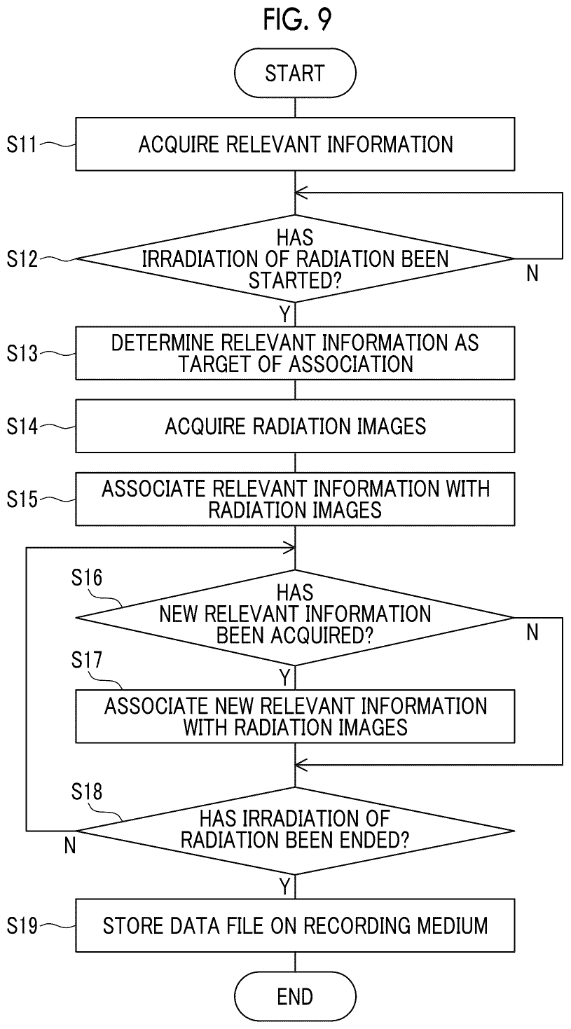

FIG. 9 is a flowchart showing an example of a flow of processing performed by the CPU 101 according to the second embodiment executing the association program 110.

In Step S11, the relevant information acquisition unit 12 acquires relevant information generated by the voice recognition apparatus 40. In a case in which a plurality of pieces of relevant information are generated intermittently by the voice recognition apparatus 40, the relevant information acquisition unit 12 acquires these pieces of relevant information in sequence.

In Step S12, the association processing unit 13 determines whether or not irradiation with radiation has been started in the radiography apparatus 20. In a case in which the radiation switch signal transitions from a low level to a high level, the association processing unit 13 determines that irradiation with radiation has started.

In Step S13, the association processing unit 13 determines the latest relevant information acquired before the start of irradiation with radiation as relevant information to be associated with a series of radiation images.

In Step S14, the image acquisition unit 11 acquires a series of radiation images captured by performing continuous irradiation with radiation in the radiography apparatus 20. A series of radiation images are obtained by fluoroscopically imaging the process in which an examinee swallows a sample in a videofluoroscopic examination of swallowing. The acquisition of a series of radiation images by the image acquisition unit 11 continues until the irradiation with radiation (fluoroscopy) ends in the radiography apparatus 20.

In Step S15, the association processing unit 13 associates the relevant information determined as the target of association in Step S13 with the radiation images (frames) acquired so far.

In Step S16, the association processing unit 13 determines whether or not new relevant information has been acquired by the relevant information acquisition unit 12 after the start of radiation. The new relevant information is relevant information generated by the voice recognition apparatus 40 by recognizing the voice uttered during the imaging period of a series of radiation images. In a case in which it is determined that new relevant information has been acquired, the process proceeds to Step S17, and in a case in which it is determined that new relevant information has not been acquired, the process proceeds to Step S18.

In Step S17, the association processing unit 13 associates the new relevant information acquired in Step S16 with the radiation images (frames) acquired after the acquisition of the new relevant information.

In Step S18, the association processing unit 13 determines whether or not the irradiation with radiation has been ended in the radiography apparatus 20. In a case in which the radiation switch signal transitions from a high level to a low level, the association processing unit 13 determines that irradiation with radiation has been ended. In a case in which it is determined in Step S18 that the irradiation with radiation has not been ended, the process returns to Step S16. Accordingly, every time new relevant information is acquired during the imaging period of a series of radiation images, the latest relevant information is associated with the radiation image (frame) acquired after the acquisition of the latest relevant information. In a case in which it is determined in Step S18 that the irradiation with radiation has been ended, the process proceeds to Step S19.

In Step S19, the association processing unit 13 generates a data file in which the most recently acquired relevant information is associated with each radiation image (each frame), and stores the data file in the recording medium.

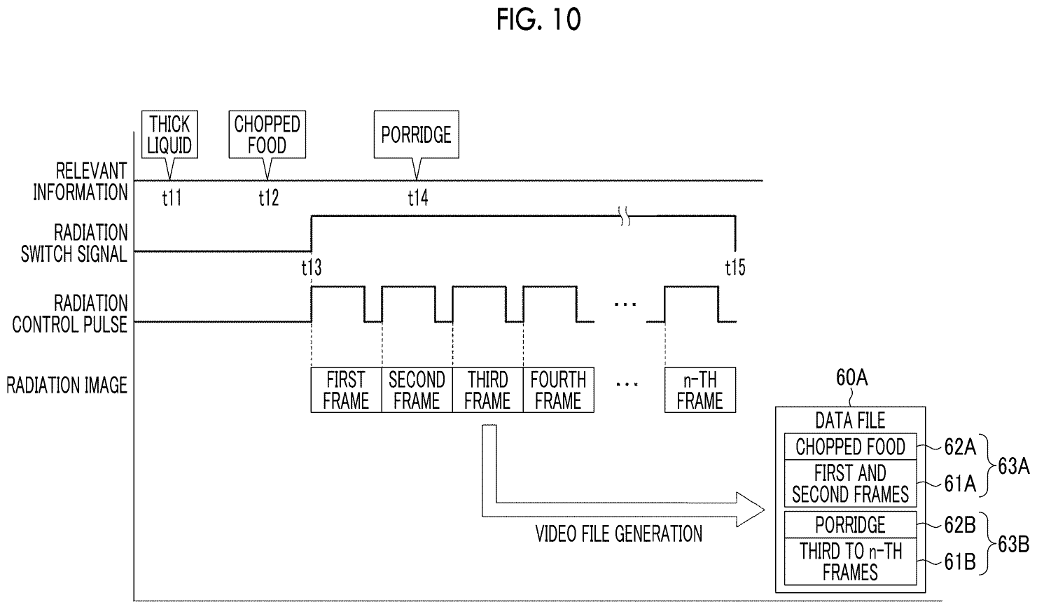

FIG. 10 is a diagram showing an example of association processing performed by the information processing apparatus 10A according to the second embodiment. In a case in which a doctor or a radiologist performing a videofluoroscopic examination of swallowing makes an utterance including the term “thick liquid” indicating the content of the examination level at time point t11, the voice recognition apparatus 40 generates identification information of the examination level corresponding to “thick liquid” (for example, text describing “thick liquid”) as relevant information. The relevant information is acquired immediately by the information processing apparatus 10A.

In a case in which as a doctor or a radiologist makes an utterance including the term “chopped food” indicating the content of the examination level at time point t12, the voice recognition apparatus 40 generates identification information of the examination level corresponding to “chopped food” (for example, text describing “chopped food”) as relevant information. The relevant information is acquired immediately by the information processing apparatus 10A.

At time point t13 at which the radiation switch signal transitions from a low level to a high level, the radiography apparatus 20 starts irradiating with radiation. In a videofluoroscopic examination of swallowing, a series of radiation images is captured by performing continuous irradiation with radiation. A series of radiation images constitute each frame in a case in which the series of radiation images are displayed as a video. A plurality of radiation images from the first frame to the n-th frame are generated in the radiography apparatus 20 until time point t15 at which the radiation switch signal transitions from a high level to a low level. During this period, irradiation with pulsed radiation is performed a plurality of times in accordance with the radiation control pulse. One radiation image (one frame) is generated by performing irradiation with one pulse of radiation. That is, each frame is generated in synchronization with a radiation control pulse.

In a case in which a doctor or a radiologist makes an utterance including the term “porridge” indicating the content of the examination level at time point t14, which is during the imaging period of a series of radiation images, the voice recognition apparatus 40 generates identification information of the examination level corresponding to “porridge” (for example, text describing “porridge”) as new relevant information. The new relevant information is acquired immediately by the information processing apparatus 10A.

The information processing apparatus 10A determines the identification information of the examination level corresponding to “chopped food”, which is the latest relevant information acquired up to time point t13 at which the radiation switch signal transitions to a high level, as relevant information corresponding to each of the radiation images (the first frame and the second frame) acquired up to time point t14 at which new relevant information is acquired, and corresponds these to each other.

The information processing apparatus 10A determines the identification information of the examination level corresponding to “porridge”, which is new relevant information acquired at time point t14 during the imaging period of the radiation images, as the relevant information corresponding to each of the radiation images acquired after time point t14 (from the third frame to the n-th frame), and corresponds these to each other.

The information processing apparatus 10A generates a data file 60A including a data block 63A including image group data 61A including some of the series of radiation images (the first frame and the second frame) and relevant information 62A corresponding to “chopped food”, and a data block 63B including image group data 61B including other some of the series of radiation images (the third frame to the n-th frame) and relevant information 62B corresponding to “porridge”, and stores this in a recording medium.

As described above, the information processing apparatus 10A according to the second embodiment of the disclosed technology associates, in a case in which new relevant information generated by recognizing a voice uttered during an imaging period of the series of radiation images is acquired, the new relevant information with a radiation image acquired after the acquisition of the new relevant information from among the series of radiation images.

In a videofluoroscopic examination of swallowing, a series of radiation images may be captured while the examination level is changed sequentially. In this case, it is necessary to be able to ascertain which frame of a video composed of a series of radiation images corresponds to which examination level. With the information processing apparatus 10A according to the present embodiment, in a case in which new relevant information generated by recognizing a voice uttered during an imaging period of the series of radiation images is acquired, a radiation image acquired after the acquisition of the new relevant information from among the series of radiation images is associated with the new relevant information. Therefore, the user does not need to perform any special operations to associate each frame with relevant information.

### Third Embodiment

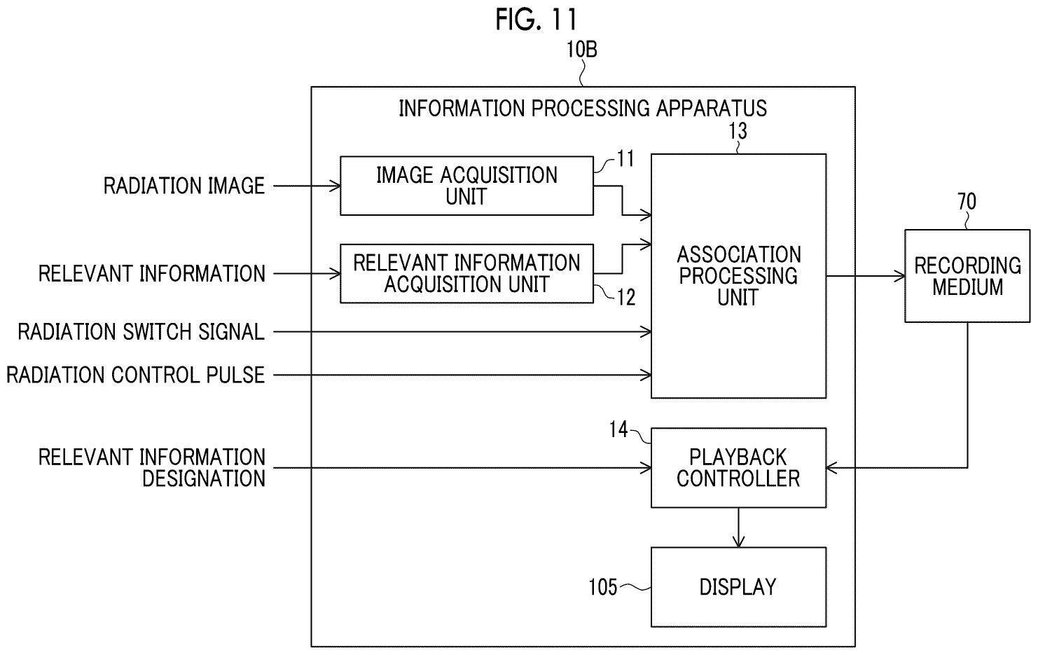

FIG. 11 is a functional block diagram showing an example of a functional configuration of an information processing apparatus 10B according to a third embodiment of the disclosed technology. Similarly to the information processing apparatus 10A according to the second embodiment described above, the information processing apparatus 10B has a function of associating different relevant information with each frame of a video composed of a series of radiation images. The information processing apparatus 10B according to the third embodiment further includes a playback controller 14. The playback controller 14 displays a radiation image associated with designated relevant information from among the series of radiation images.

A data file in which certain relevant information is associated with some of frames of a video composed of a series of radiation images and different relevant information is associated with some of the other frames is generated by the association processing unit 13, and this data file is stored in a recording medium 70. The recording medium 70 may be a non-volatile memory 103 included in the information processing apparatus 10B.

The user operates the input device 104 to issue an instruction to the information processing apparatus 10B to play back the data file stored in the recording medium 70 together with the designation of relevant information. The user can designate a portion (frame) that he/she wishes to play back from a video composed of a series of radiation images by designating the relevant information. For example, in a case of trying to play back a portion (frame) associated with identification information corresponding to “chopped food” as relevant information, the user issues an instruction to play back the data file by designating “chopped food”. The playback controller 14 reads out the data file from the recording medium 70 in response to the playback instruction, and plays back the image group data included in the data file that has been associated with relevant information corresponding to the “chopped food”. On the display 105, among the series of radiation images, some radiation images, which are associated with relevant information corresponding to the “chopped food”, are displayed as a video.

As described above, with the information processing apparatus 10B according to the third embodiment of the disclosed technology, in the case of playing back a video composed of a series of radiation images captured while sequentially changing the examination level, it becomes possible to easily play back the portion (frame) corresponding to the desired examination level.

In each of the above embodiments, a case in which the disclosed technology is applied to a radiation image acquired in a videofluoroscopic examination of swallowing has been described as an example, but the disclosed technology is not limited to this aspect. The disclosed technology can be applied to any radiation images acquired in examinations, diagnoses, or the like other than videofluoroscopic examinations of swallowing.

In addition, in each of the above embodiments, a configuration in which the radiography system 1 comprises the voice recognition apparatus 40 has been described as an example, but each of the information processing apparatus 10, 10A, and 10B may have at least a part of the functions of the voice recognition apparatus 40. For example, the voice recognition apparatus 40 may transmit a voice recognition result to the information processing apparatus 10, and the information processing apparatus 10 may generate relevant information based on the recognized voice. Furthermore, the information processing apparatuses 10, 10A, and 10B may recognize a voice and generate relevant information based on the recognized voice.

As hardware for executing processes in each functional unit of the information processing apparatuses 10, 10A, and 10B, various processors as shown below can be used. The processor may be a CPU that executes software (programs) and functions as various processing units. Furthermore, the processor may be a programmable logic device (PLD) such as an FPGA whose circuit configuration is changeable. Moreover, the processor may have a circuit configuration that is specially designed to execute a specific process, such as an application-specific integrated circuit (ASIC).

Each functional unit of the information processing apparatuses 10, 10A, and 10B may be configured by one of the various processors described above, or may be configured by a combination of the same or different kinds of two or more processors (for example, a combination of a plurality of FPGAs or a combination of the CPU and the FPGA). In addition, a plurality of functional units may be configured by one processor.

In the above embodiments, the association program 110 has been described as being stored (installed) in the non-volatile memory 103 in advance; however, the present disclosure is not limited thereto. The association program 110 may be provided in a form recorded in a recording medium such as a compact disc read-only memory (CD-ROM), a digital versatile disc read-only memory (DVD-ROM), and a universal serial bus (USB) memory. In addition, the association program 110 may be configured to be downloaded from an external device via a network.

Regarding the first to third embodiments, the following supplementary notes are further disclosed.

### Supplementary Note 1

An information processing apparatus comprising at least one processor,
in which the processor is configured to:
   acquire a series of radiation images captured by performing continuous irradiation with radiation;
   acquire relevant information that is generated by recognizing a voice and is related to imaging content of the series of radiation images; and
   associate the series of radiation images with the relevant information.

### Supplementary Note 2

The information processing apparatus according to Supplementary Note 1,
in which the processor is configured to associate the latest relevant information acquired before a predetermined timing synchronized with the capturing of the series of radiation images with the series of radiation images.

### Supplementary Note 3

The information processing apparatus according to Supplementary Note 2,
in which the predetermined timing is a timing of start of irradiation with the radiation that is continuously performed in the capturing of the series of radiation images.

### Supplementary Note 4

The information processing apparatus according to Supplementary Note 3,
in which the predetermined timing is confirmed by receiving a radiation switch signal, which is transmitted by a user input unit and indicates the start of irradiation that is continuously performed in the capturing of the series of radiation images.

### Supplementary Note 5

The information processing apparatus according to any one of Supplementary Notes 1 to 4,
in which the processor is configured to generate a data file including the relevant information and video data including the series of radiation images.

### Supplementary Note 6

The information processing apparatus according to any one of Supplementary Notes 1 to 5,
in which the processor includes a notation corresponding to the relevant information in a file name of a data file including the series of radiation images.

### Supplementary Note 7

The information processing apparatus according to any one of Supplementary Notes 1 to 6,
in which the processor is configured to process video data including the series of radiation images so that a notation corresponding to the relevant information is displayed together with the series of radiation images.

### Supplementary Note 8

The information processing apparatus according to any one of Supplementary Notes 1 to 7,
in which the processor is configured to associate, in a case in which new relevant information generated by recognizing a voice uttered during an imaging period of the series of radiation images is acquired, the new relevant information with a radiation image acquired after the acquisition of the new relevant information from among the series of radiation images.

### Supplementary Note 9

The information processing apparatus according to any one of Supplementary Notes 1 to 8,
in which the processor is configured to display a radiation image associated with designated relevant information from among the series of radiation images.

### Supplementary Note 10

The information processing apparatus according to any one of Supplementary Notes 1 to 9,
in which the processor is configured to:
   acquire relevant information generated by a method other than voice recognition; and
   replace the relevant information generated by the voice recognition with the relevant information generated by the method other than the voice recognition.

### Supplementary Note 11

The information processing apparatus according to any one of Supplementary Notes 1 to 10,
in which the processor is configured to issue an alert in a case in which the relevant information associated with the series of radiation images does not include specific information.

### Supplementary Note 12

The information processing apparatus according to any one of Supplementary Notes 1 to 11,
in which the relevant information is information indicating a type of sample used in an examination using the series of radiation images.

### Supplementary Note 13

The information processing apparatus according to any one of Supplementary Notes 1 to 12,
in which the relevant information is information indicating a type of posture of an examinee in an examination using the series of radiation images.

### Supplementary Note 14

The information processing apparatus according to Supplementary Note 12 or 13, in which the examination is a videofluoroscopic examination of swallowing.

### Supplementary Note 15

The information processing apparatus according to Supplementary Note 1,
in which the relevant information is information indicating a type of imaging direction of the series of radiation images.

### Supplementary Note 16

The information processing apparatus according to any one of Supplementary Note 1 to Supplementary Note 15,
in which the relevant information is generated by recognizing a voice by which a predetermined term is pronounced.

### Supplementary Note 17

An information processing method executed by at least one processor included in an information processing apparatus, the information processing method comprising:
acquiring a series of radiation images captured by performing continuous irradiation with radiation;
acquiring relevant information that is generated by recognizing a voice and is related to imaging content of the series of radiation images; and
associating the series of radiation images with the relevant information.

### Supplementary Note 18

A program for causing at least one processor included in an information processing apparatus to execute:
acquiring a series of radiation images captured by performing continuous irradiation with radiation;
acquiring relevant information that is generated by recognizing a voice and is related to imaging content of the series of radiation images; and
associating the series of radiation images with the relevant information.

What is claimed is:

1. An information processing apparatus comprising at least one processor,
wherein the processor is configured to:
acquire a series of radiation images captured by performing continuous irradiation with radiation;
acquire relevant information that is generated by recognizing a voice and is related to imaging content of the series of radiation images; and
associate the series of radiation images with the relevant information.

2. The information processing apparatus according to claim 1,
wherein the processor is configured to associate the latest relevant information acquired before a predetermined timing synchronized with the capturing of the series of radiation images with the series of radiation images.

3. The information processing apparatus according to claim 2,
wherein the predetermined timing is a timing of start of irradiation with the radiation that is continuously performed in the capturing of the series of radiation images.

4. The information processing apparatus according to claim 3,
wherein the predetermined timing is confirmed by receiving a radiation switch signal, which is transmitted by a user input unit and indicates the start of irradiation that is continuously performed in the capturing of the series of radiation images.

5. The information processing apparatus according to claim 1,
wherein the processor is configured to generate a data file including the relevant information and image group data including the series of radiation images.

6. The information processing apparatus according to claim 1,
wherein the processor includes a notation corresponding to the relevant information in a file name of a data file including the series of radiation images.

7. The information processing apparatus according to claim 1,
wherein the processor is configured to process image group data including the series of radiation images so that a notation corresponding to the relevant information is displayed together with the series of radiation images.

8. The information processing apparatus according to claim 1,
wherein the processor is configured to associate, in a case in which new relevant information generated by recognizing a voice uttered during an imaging period of the series of radiation images is acquired, the new relevant information with a radiation image acquired after the acquisition of the new relevant information from among the series of radiation images.

9. The information processing apparatus according to claim 1,
wherein the processor is configured to display a radiation image associated with designated relevant information from among the series of radiation images.

10. The information processing apparatus according to claim 1,
wherein the processor is configured to:
acquire relevant information generated by a method other than voice recognition; and
replace the relevant information generated by the voice recognition with the relevant information generated by the method other than the voice recognition.

11. The information processing apparatus according to claim 1,
wherein the processor is configured to issue an alert in a case in which the relevant information associated with the series of radiation images does not include specific information.

12. The information processing apparatus according to claim 1,
wherein the relevant information is information indicating a type of sample used in an examination using the series of radiation images.

13. The information processing apparatus according to claim 1,
wherein the relevant information is information indicating a type of posture of an examinee in an examination using the series of radiation images.

14. The information processing apparatus according to claim 12,
wherein the examination is a videofluoroscopic examination of swallowing.

15. The information processing apparatus according to claim 1,
wherein the relevant information is information indicating a type of imaging direction of the series of radiation images.

16. The information processing apparatus according to claim 1,
wherein the relevant information is generated by recognizing a voice by which a predetermined term is pronounced.

17. An information processing method executed by at least one processor included in an information processing apparatus, the information processing method comprising:
acquiring a series of radiation images captured by performing continuous irradiation with radiation;
acquiring relevant information that is generated by recognizing a voice and is related to imaging content of the series of radiation images; and
associating the series of radiation images with the relevant information.

18. A non-transitory computer-readable storage medium storing a program for causing at least one processor included in an information processing apparatus to execute:
acquiring a series of radiation images captured by performing continuous irradiation with radiation;
acquiring relevant information that is generated by recognizing a voice and is related to imaging content of the series of radiation images; and
associating the series of radiation images with the relevant information.
